(19) United States
(12) Patent Application Publication
(10) Pub. No.: US 2025/0366872 A1
(43) Pub. Date: Dec. 4, 2025

(54) CUTTING GUIDES AND ASSOCIATED METHODS FOR ORTHOPEDIC PROCEDURES

(71) Applicant: Paragon 28, Inc., Englewood, CO (US)

(72) Inventors: Lucas Charles MCMAHAN, Lone Tree, CO (US); Michael SCHMIDT, Greenwood Village, CO (US)

(73) Assignee: Paragon 28, Inc., Englewood, CO (US)

(21) Appl. No.: 19/219,419

(22) Filed: May 27, 2025

Related U.S. Application Data
(60) Provisional application No. 63/652,261, filed on May 28, 2024.

(51) Int. Cl. A61B 17/17; A61B 17/00

(52) U.S. Cl. CPC A61B 17/1775; A61B 2017/00367; A61B 2560/04

(57) ABSTRACT

An orthopedic instrument system includes a first cutting guide. The first cutting guide includes a base portion having an opening, an extension extending from the base portion having a cannulated opening in fluid communication with the opening of the base portion, and an actuator configured to occupy at least a portion of the opening of the base when in an actuated position within the cannulated opening. The system also includes a cutting insert configured to be received within the opening. The insert includes at least one depression, wherein the at least one depression comprises a volume which, when positioned within the opening, is common with at least a portion of the volume of the opening occupied by the actuator when in the actuated position.

### CROSS RELATED APPLICATIONS

This application claims benefit of priority of U.S. Provisional Application No. 63/652,261 filed on May 28, 2024, and entitled “Cutting Guides and Associated Methods For Orthopedics Procedures,” the disclosure of which is hereby incorporated herein by reference in its entirety.

### TECHNICAL FIELD

The present disclosure relates to systems, instruments, and surgical methods to be implemented in surgical procedures. The present disclosure relates to podiatric and instruments and surgical methods to be implemented in various procedures of the foot, ankle, or other anatomy. More specifically, but not exclusively, the present disclosure relates to a system and associated methodology to be implemented procedures of the foot and/or ankle.

The present disclosure relates to systems, instruments, and surgical methods to be implemented in surgical procedures. The present disclosure relates to podiatric and instruments and surgical methods to be implemented in various procedures of the foot, ankle, or other anatomy. More specifically, but not exclusively, the present disclosure relates to a system and associated methodology to be implemented procedures of the foot and/or ankle.

### BACKGROUND OF THE INVENTION

Many currently available surgical systems, instruments, and methods do not completely address the needs of patients. Additionally, many currently available surgical systems, instruments, and methods fail to account for properties of foot and ankle anatomy and accordingly can decrease favorability of the outcome for the patient.

### SUMMARY OF THE INVENTION

The present disclosure is directed toward systems, instruments, and surgical methods to be implemented in procedures of the foot and ankle.

One aspect of the present disclosure is directed to an orthopedic instrument system that includes a first cutting guide. The first cutting guide includes a base portion having an opening, an extension extending from the base portion having a cannulated opening in fluid communication with the opening of the base portion, and an actuator configured to occupy at least a portion of the opening of the base when in an actuated position within the cannulated opening. The system also includes a cutting insert configured to be received within the opening. The insert includes at least one depression, wherein the at least one depression comprises a volume which, when positioned within the opening, is common with at least a portion of the volume of the opening occupied by the actuator when in the actuated position.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is an elevated, front, perspective view of an exemplary orthopedic system, in accordance with the present disclosure;

FIG. 2 is an elevated, rear, perspective view of the exemplary orthopedic system of FIG. 1, in accordance with the present disclosure;

FIG. 3 is a front view of the exemplary orthopedic system of FIG. 1, in accordance with the present disclosure;

FIG. 4 is a top view of components of the exemplary orthopedic system of FIG. 1, in accordance with the present disclosure;

FIG. 5 is an alternate front view of the components of FIG. 4 of the exemplary orthopedic system of FIG. 1, in accordance with the present disclosure;

FIG. 6 is a rear view of the exemplary orthopedic system of FIG. 1, in accordance with the present disclosure;

FIG. 7 is a front perspective view of the components of FIG. 4 of the exemplary orthopedic system of FIG. 1, in accordance with the present disclosure;

FIG. 8 is a rear perspective view of the components of FIG. 4 of the exemplary orthopedic system of FIG. 1, in accordance with the present disclosure;

FIG. 9 is a top view of the components of FIG. 4 of the exemplary orthopedic system of FIG. 1, in accordance with the present disclosure;

FIG. 10 is a bottom view of the components of FIG. 4 of the exemplary orthopedic system of FIG. 1, in accordance with the present disclosure;

FIG. 11 is a side view of the components of FIG. 4 of the exemplary orthopedic system of FIG. 1, in accordance with the present disclosure;

FIG. 12 is a side view of the components of FIG. 4 of the exemplary orthopedic system of FIG. 1, in accordance with the present disclosure;

FIG. 13 is a front view of the components of FIG. 4 of the exemplary orthopedic system of FIG. 1, in accordance with the present disclosure;

FIG. 14 is a rear view of the components of FIG. 4 of the exemplary orthopedic system of FIG. 1, in accordance with the present disclosure;

FIG. 15 is a side view of an additional component implemented in conjunction with the exemplary orthopedic system of FIG. 1, in accordance with the present disclosure;

FIG. 16 is a perspective view of the additional component of FIG. 15, in accordance with the present disclosure;

FIG. 17 is a front perspective view of a component of the exemplary orthopedic system of FIG. 1, in accordance with the present disclosure;

FIG. 18 is a rear perspective view of the component of FIG. 17 of the exemplary orthopedic system of FIG. 1, in accordance with the present disclosure;

FIG. 19 is a front view of the component of FIG. 17 of the exemplary orthopedic system of FIG. 1, in accordance with the present disclosure;

FIG. 20 is a rear view of the component of FIG. 17 of the exemplary orthopedic system of FIG. 1, in accordance with the present disclosure;

FIG. 21 is a top view of the component of FIG. 17 of the exemplary orthopedic system of FIG. 1, in accordance with the present disclosure;

FIG. 22 is a bottom view of the component of FIG. 17 of the exemplary orthopedic system of FIG. 1, in accordance with the present disclosure;

FIG. 23 is a side view of the component of FIG. 17 of the exemplary orthopedic system of FIG. 1, in accordance with the present disclosure;

FIG. 24 is an alternate side view of the component of FIG. 17 of the exemplary orthopedic system of FIG. 1, in accordance with the present disclosure;

FIG. 25 is an elevated, front, perspective view of an exemplary orthopedic system, in accordance with the present disclosure;

FIG. 26 is an elevated, side, perspective view of the exemplary orthopedic system of FIG. 25, in accordance with the present disclosure;

FIG. 27 is a side view of the exemplary orthopedic system of FIG. 25, in accordance with the present disclosure;

FIG. 28 is a top view of the components of the exemplary orthopedic system of FIG. 25, in accordance with the present disclosure;

FIG. 29 is an alternate side view of the components of FIG. 28 of the exemplary orthopedic system of FIG. 25, in accordance with the present disclosure;

FIG. 30 is a rear view of the components of FIG. 28 of the exemplary orthopedic system of FIG. 1, in accordance with the present disclosure;

FIG. 31 is a front perspective view of the components of FIG. 28 of the exemplary orthopedic system of FIG. 25, in accordance with the present disclosure;

FIG. 32 is a rear perspective view of the components of FIG. 28 of the exemplary orthopedic system of FIG. 25, in accordance with the present disclosure;

FIG. 33 is a top view of the components of FIG. 28 of the exemplary orthopedic system of FIG. 25, in accordance with the present disclosure;

FIG. 34 is a bottom view of the components of FIG. 28 of the exemplary orthopedic system of FIG. 25, in accordance with the present disclosure;

FIG. 35 is a side view of the components of FIG. 28 of the exemplary orthopedic system of FIG. 25, in accordance with the present disclosure;

FIG. 36 is an alternative side view of the components of FIG. 28 of the exemplary orthopedic system of FIG. 25, in accordance with the present disclosure;

FIG. 37 is a front view of the components of FIG. 28 of the exemplary orthopedic system of FIG. 25, in accordance with the present disclosure; and

FIG. 38 is a rear view of the components of FIG. 28 of the exemplary orthopedic system of FIG. 25, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

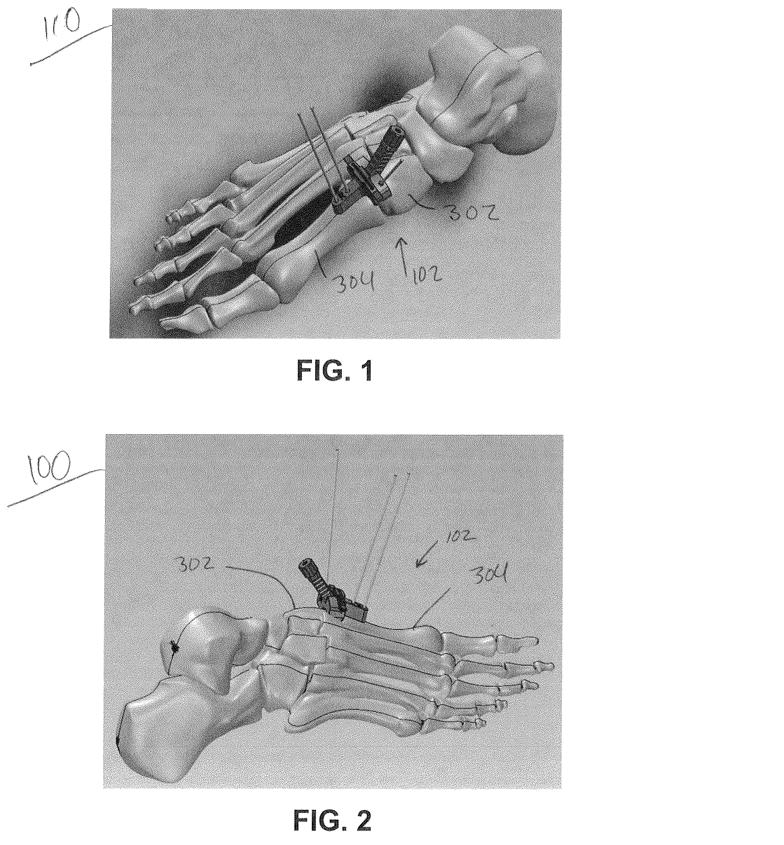

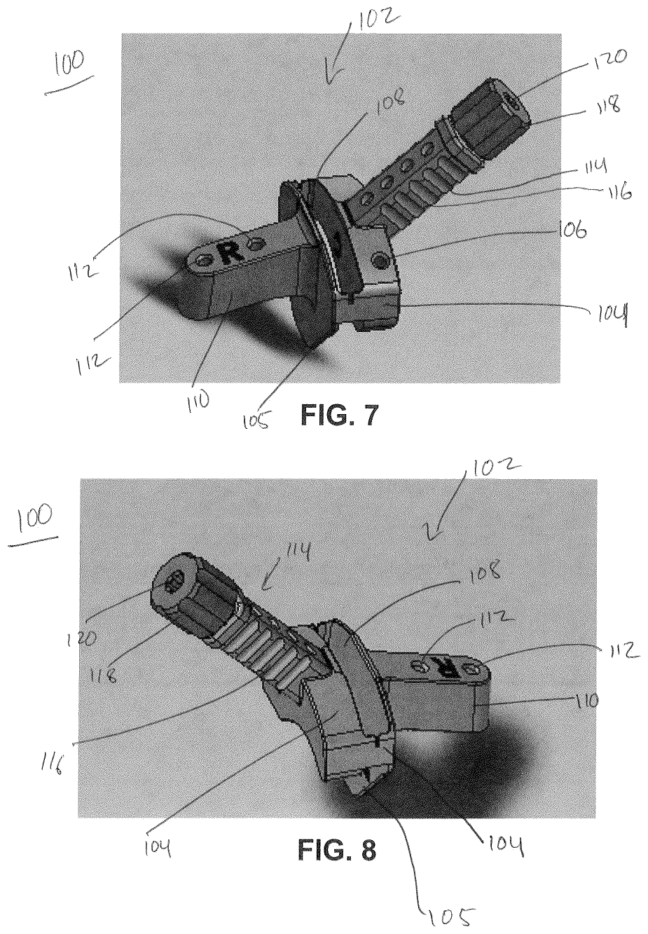

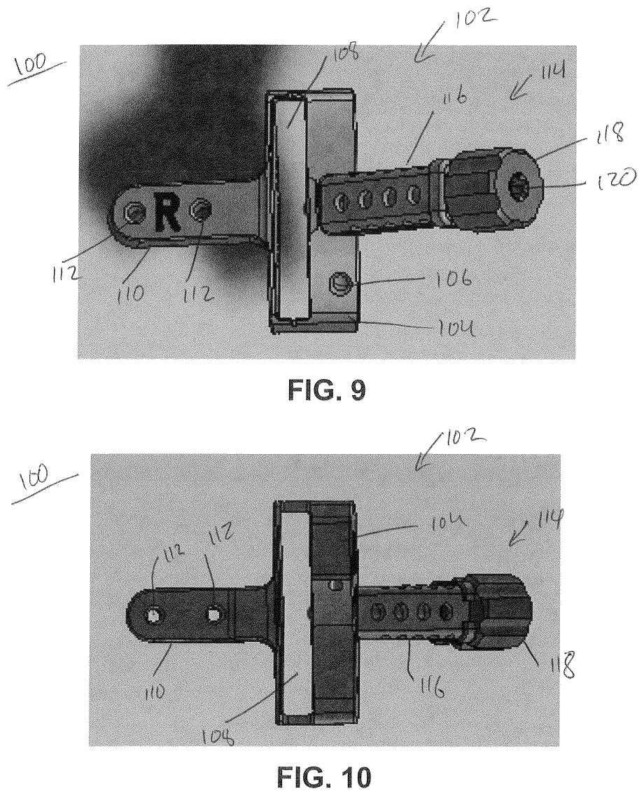

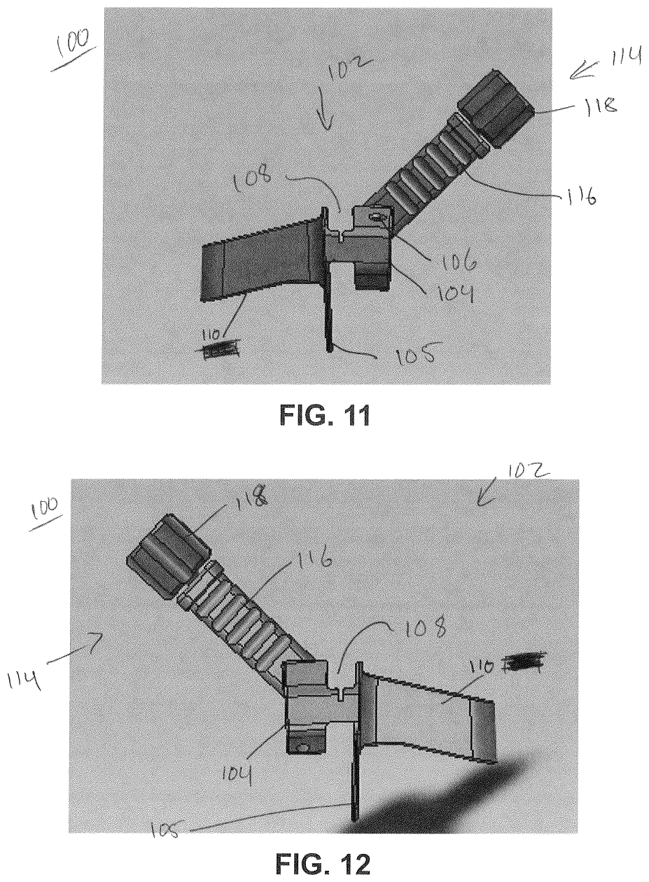

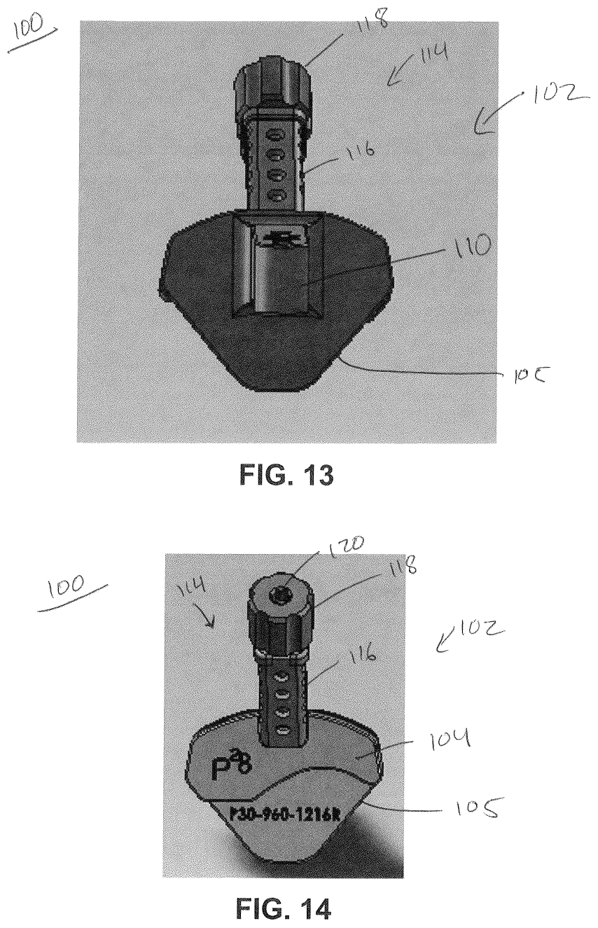

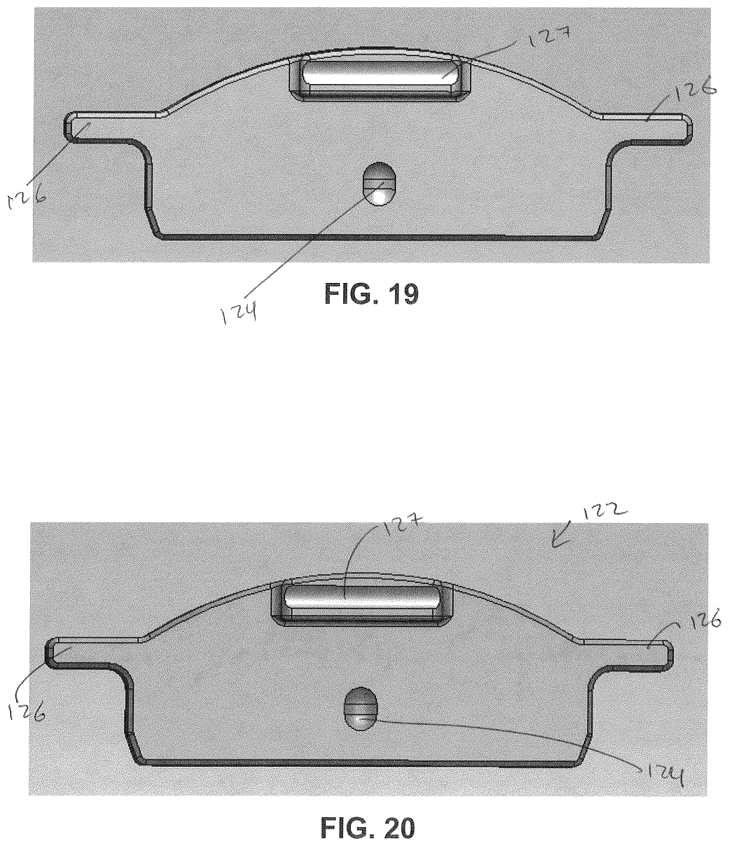

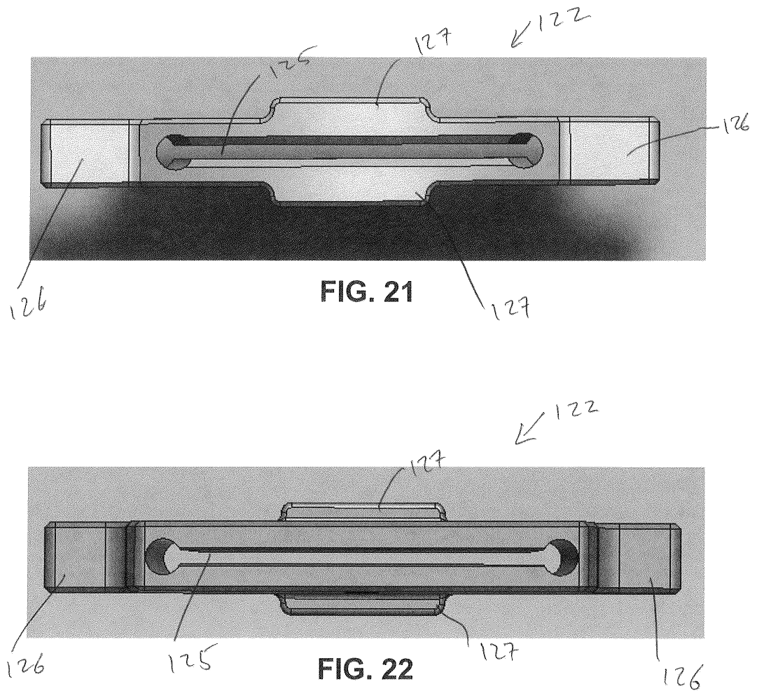

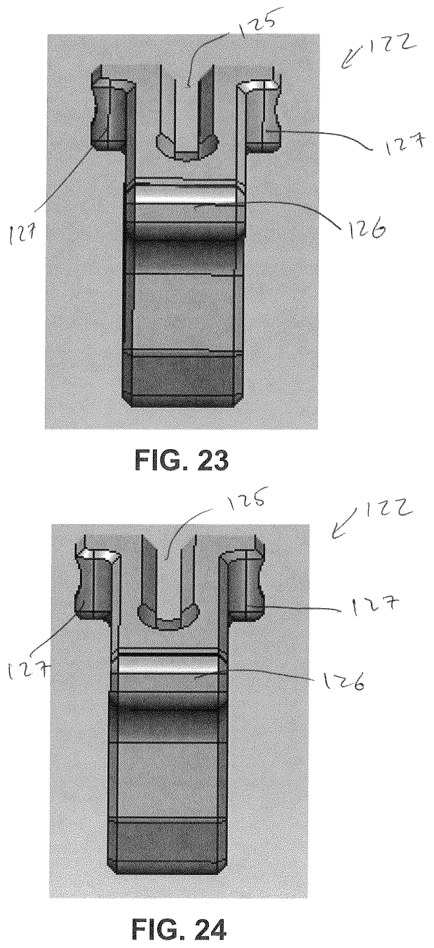

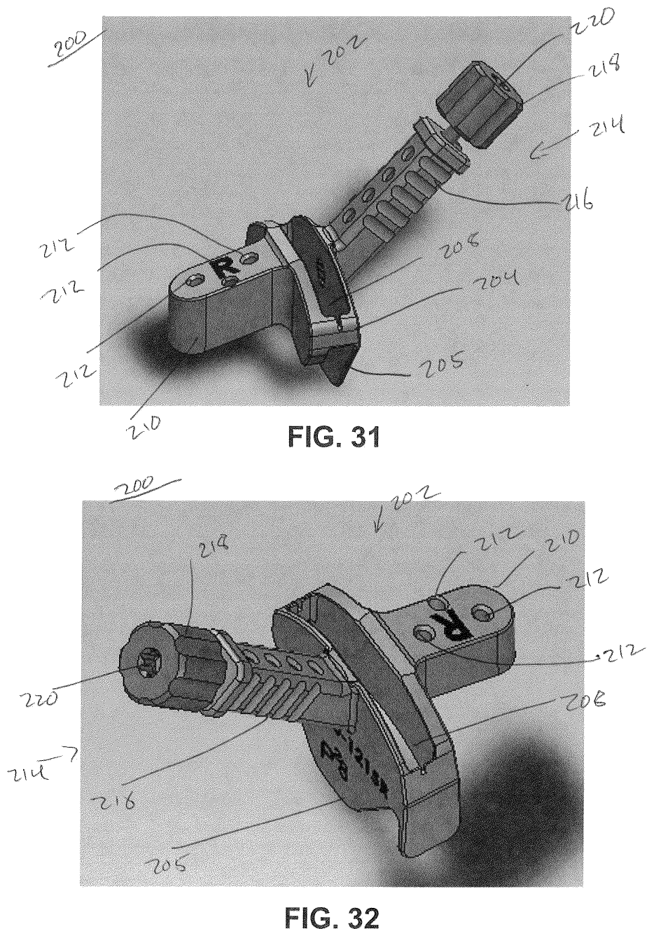

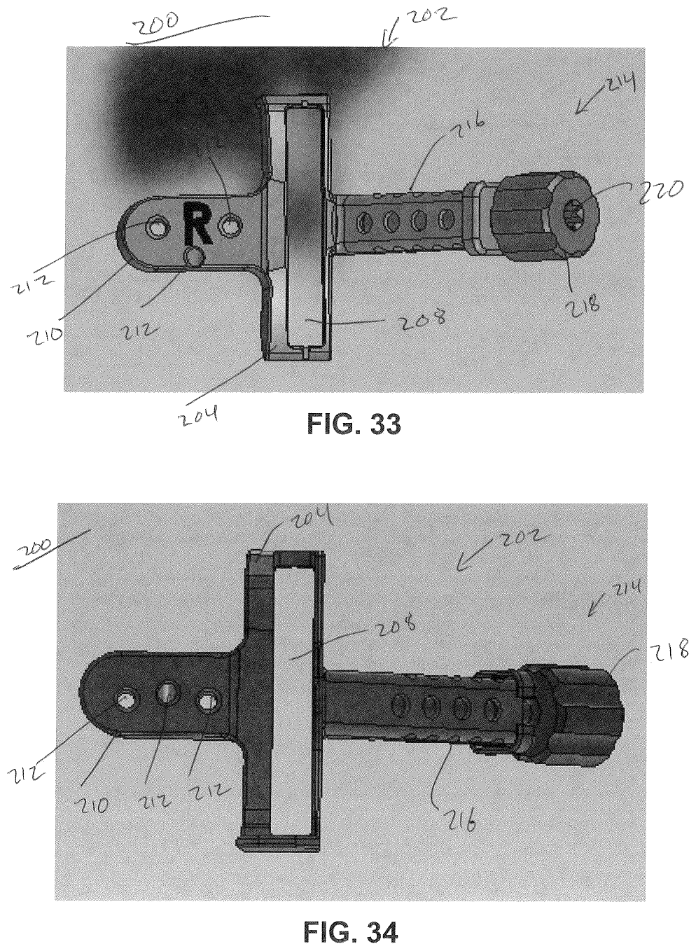

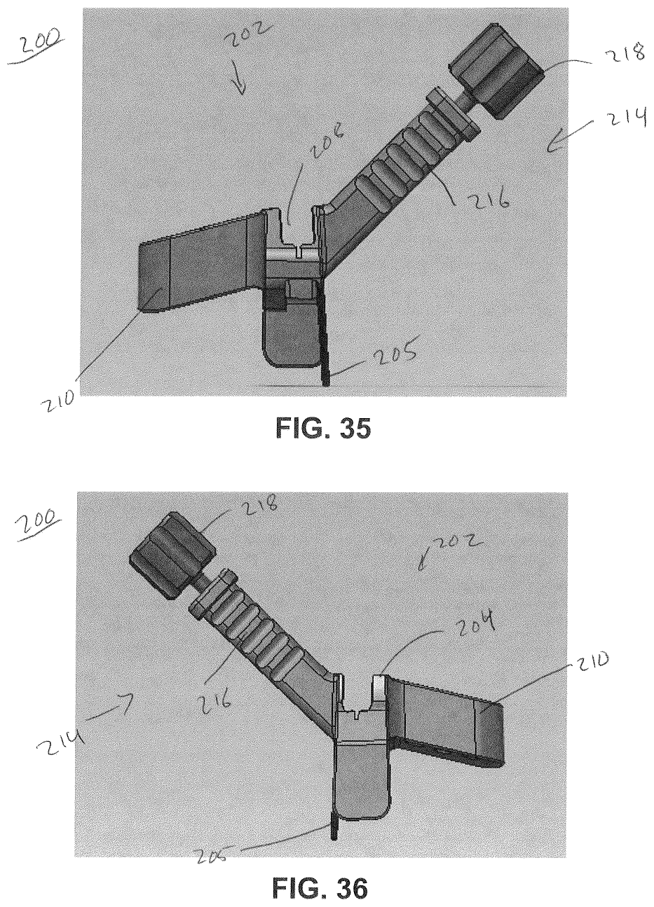

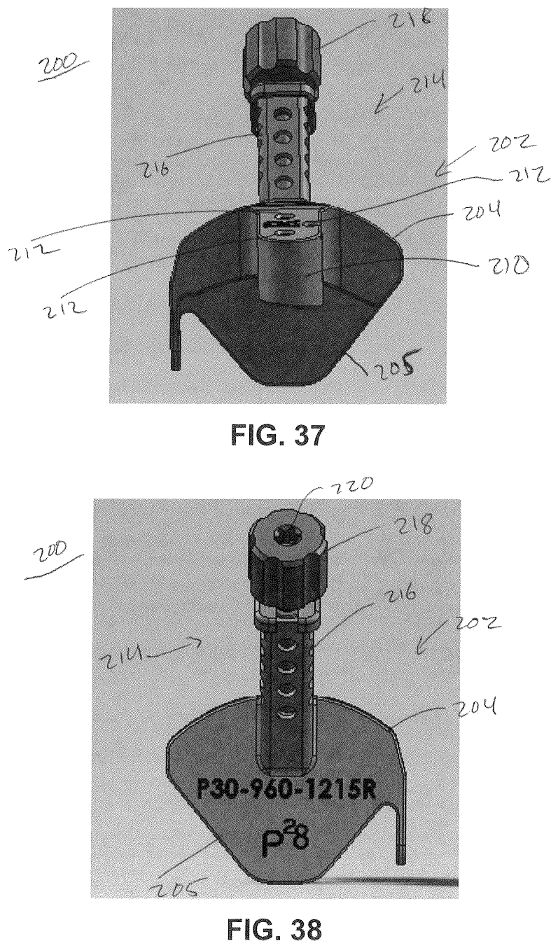

Referring now to FIGS. 1-38, a system 100 and a system 200 and components thereof are shown, according to exemplary embodiments. In some aspects, the systems 100, 200 may be subsystems of a larger surgical system or portion thereof and, accordingly, may both be implemented in a surgical procedure. However, one or both of the systems 100, 200 may be singularly implemented in a surgical procedure. In some aspects, one or more components may be included and/or compatible with both of the systems 100, 200, and may be releasably couplable/decouplable with one or more components of said systems 100, 200.

Referring now to FIGS. 1-16, the system 100 and components thereof are shown, according to an exemplary embodiment. The system 100 is shown to include a cutting guide 102 (referred to hereinafter as “guide 102”), which is shown to be coupled on a proximal side thereof with a medial cuneiform 302 of a patient and on a distal side with a first metatarsal 304 of a patient. The guide 102 may be releasably couplable with one or more bones of the anatomy, for example the medial cuneiform 302 and first metatarsal 304, by inserting one or more k-wires through openings in the guide 102 having a complimentary geometry to said k-wires, with the k-wires then advanced into the aforementioned bony anatomy.

The guide 102 is shown to include a base 104 which, as shown, includes a substantially rectangular footprint. In some aspects, the base 104 may include alternate geometries and, accordingly, alternate footprints (e.g., oval, pentagonal, hexagonal, etc.). In some aspects, the bottom surface of the base may include a curvature along at least a portion thereof configured to accommodate at least a portion of a contour of the first metatarsal 304 and/or the medial cuneiform 302. The base 104 is shown to include an opening 106 positioned on a proximal (and medial) side thereof and extend from a top surface of the base 104 through to a bottom side of the base 104, establishing fluid communication therebetween. As shown in FIGS. 1-6, the opening 106 may be configured to receive a k-wire to facilitate releasable coupling with the medial cuneiform 302 of the patient. In some aspects, the base 104 may include multiple openings 106, for example positioned adjacent one another or on opposite sides of the base 104.

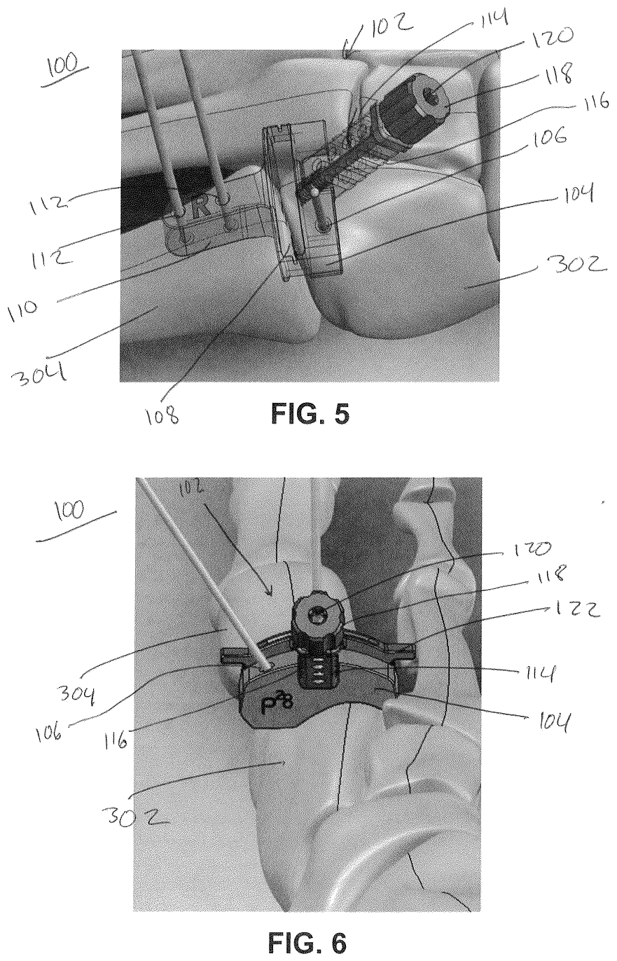

The base 104 is further shown to include an opening 108 positioned distally relative to the opening 106 and, like the opening 106, extending from the top surface through to the bottom surface of the base 104 thus establishing fluid communication therebetween. In some aspects, the base 104 may be configured to receive one or more components therein (e.g., releasably couple with/retain said components). Further, in some aspects the opening 108 may be configured to receive at least a portion of a cutting instrument therein and therethrough, for example a saw (e.g., sagittal, reciprocating, etc.) or a burring instrument to guide cutting or otherwise modifying bony anatomy disposed beneath the opening 108. In some aspects, any such cutting or burring instruments may be manipulated through one or more slots or other openings disposed in the aforementioned inserts which may be releasably coupled with and/or retained within the opening 108. As shown, the opening 108 is positioned relative to the base 104 and the guide 102 such that, when the guide 102 is positioned as shown in at least FIG. 5, the opening 108 is positioned substantially above the first tarsometatarsal (“TMT”) joint (and more specifically, over a distal portion of the medial cuneiform 302), which is the joint at which the medial cuneiform 302 articulates at a distal portion thereof with a proximal portion of the first metatarsal 304. However, the opening 108 may also be positioned elsewhere about the base 104 and the guide 102 so as to facilitate positioning of the opening 108 relative to various bony anatomy or joints.

The base 104 includes a paddle 105 extending in a downward direction from the bottom surface of the base 104 just distally relative to the opening 108. As shown, the paddle 105 is positioned at a distal-most portion of the base 104 and may have a substantially rounded, triangular geometry (e.g., similar to that of a guitar pick) or similar. As shown in at least FIG. 4, the paddle 105 may be positioned within the first TMT joint (e.g., between the medial cuneiform 302 and the first metatarsal 304) when the guide 102 is engaged with said anatomy as shown.

The base 104 is further shown to include a projection 110 extending from the base 104 in the distal direction (e.g., just above the paddle 105). As shown, the projection 110 has a substantially elongated geometry with a rounded end, although the projection 110 may have alternate geometries in some embodiments. The projection 110 is shown to include at least one opening 112 (shown in FIGS. 1-15 as a pair of openings 112) extending from a top surface of the projection 110 through to the bottom surface and establishing fluid communication therebetween. The at least one opening 112 may be sized to receive at least one k-wire therein and therethrough so as to facilitate engagement of the projection 110 with bony anatomy (e.g., the first metatarsal 304, as shown). As shown in FIGS. 1-15, the pair of openings 112 are positioned with a footprint overlapping with a longitudinal axis of the projection 110, but in some aspects the openings 112 may be positioned otherwise. Further, the projection 110 may include one, two, or various other numbers of openings 112 positioned variously about the projection 110.

The base 104 is further shown to include an extension 114, which is shown to extend from a proximal-most portion of the base 104 at an oblique angle relative to the top surface of the base 104. In some aspects, the extension 114 may extend at an approximately 45-degree angle from the base 104, or may extend from the base 104 at other alternate angles. The extension 114 as shown includes a substantially rectangular body, which includes a texture 116 disposed on at least a portion of the outer surfaces thereof to facilitate gripping of the extension 114 by a physician. At least a portion of the extension 114 is shown to be substantially cannulated such that it may receive at least a portion of an actuator 118 (and a shaft 119 of the actuator 118) therein.

The actuator 118 includes a head portion 120, with the shaft 119 extending from the head portion 120. Both the head portion 120 and the shaft 119 may include a cannulation extending along at least a portion of a common longitudinal axis thereof. The shaft 119 includes a threading disposed on at least a portion of the outer surface thereof, shown in FIG. 5 to be positioned on a distal portion of the shaft 119. In some aspects, the extension 114 may include a threading complimentary to that of the shaft 119 within the cannulation and positioned at a distal end of the extension 114. Accordingly, the actuator 118 may be threadably coupled (and decoupled) with the extension 114 by twisting the head portion 120 so as to engage or disengage the threading of the shaft 119 with that of the extension 114. The head portion 120 of the actuator 118 may include one or more interfaces configured to facilitate engagement with one or more instruments, for example a hexalobe driver. The head portion 120 may include one or more geometric features configured to facilitate such engagement, and an instrument (such as the hexalobe driver) may be implemented by a physician in order to manipulate (e.g., loosen/tighten, advance/withdraw, etc.) the actuator 118 within the extension 114.

The system 100 is further shown to include an insert 122, which may be placed within the opening 108 of the guide 102 as shown in at least FIG. 6. The insert 122 is shown to include a depression 124 disposed on lateral surfaces thereof. When the insert 122 is positioned within the opening 108 (e.g., as shown in FIG. 6), the actuator 118 may be threadably coupled with the extension 114 and advanced via the threading of the shaft 119 along the extension 114 such that a distal-most portion of the shaft 119 contacts and is positioned within the depression 124. Accordingly, the insert 122 may be retained (e.g., in a coupled configuration) within the opening 108 by the actuator 118, as the distal portion of the shaft 119 prevents removal of the insert 122 from the opening 108 when the distal portion of the shaft 119 occupies at least a portion of the depression 124. The insert 122 may be decoupled from the guide 102 by manipulating the actuator 118 such that the distal portion of the shaft 119 vacates the depression 124, thus allowing for removal (e.g., decoupling) of the insert 122 from the opening 108.

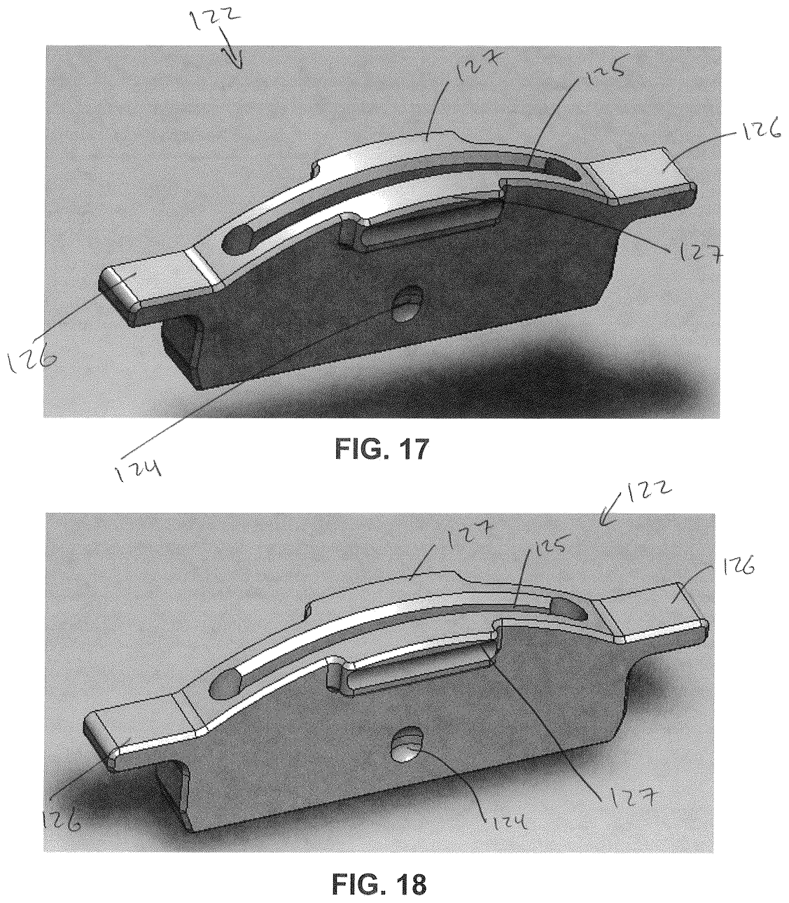

As shown in at least FIGS. 17-24, the insert 122 includes a slot 125 positioned in a central portion thereof and extending from a top surface through to a bottom surface of the insert 122, establishing fluid communication therebetween. The slot 125 may be sized to receive various orthopedic instruments therein, for example sagittal or reciprocating saws or burr devices. The insert 122 also includes a first pair of protrusions 126 positioned at opposite ends of the slot 125, and a second pair of protrusions 127 positioned centrally along the length of the slot 125 on opposite sides of the insert 122. Collectively, the first and second pair of protrusions 126, 127 establish a footprint of the insert 122 greater than the area of the opening 108 such that the insert 122 may be “dropped” within the opening 108. Accordingly, the first and second pair of protrusions 126, 127 contact a top surface of the base 104 and remain positioned above the opening 108, while a portion (for example, a majority) of the insert 122 is suspended within the opening 108. The system 100 may include one or more inserts 122 configured to accommodate different cutting instrumentation and having different sizing/dimensions/angulation.

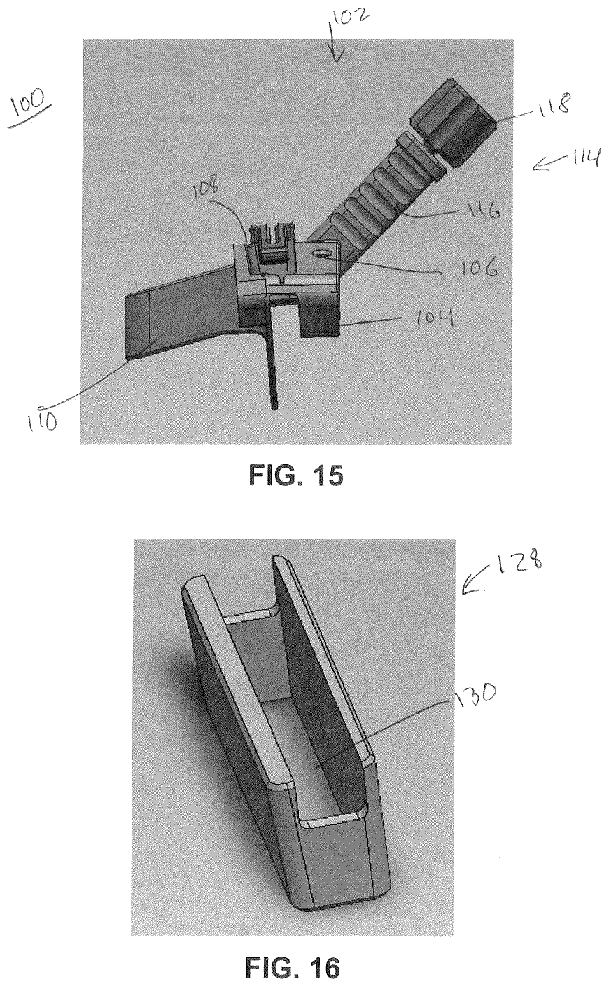

The system 100 is further shown to include an angled insert 128, shown in FIGS. 15-16 according to an exemplary embodiment. As shown in FIG. 16, the angled insert 128 is configured to be received at least partially within the opening 108 so as to provide a specified angular offset to any cut performed using the system 100. The angled insert 128 includes an opening 130 positioned in a central portion thereof and configured to receive at least a portion of the insert 122 therein. The opening 130 is shown to be offset from vertical at an oblique angle (e.g., 2 degrees, 4 degrees, 5 degrees, 6 degrees, 8 degrees, 10 degrees, or other degree measurements). Similar to the engagement of the insert 122 within the opening 108, one or both of the pair of projections 126, 127 may extend beyond the footprint of the slot 130 such that the insert 122 may be “dropped” within the slot 130 with the pair of projections 126 and/or 127 contacting the angled insert 128 and positioned above a top surface thereof. Accordingly, when placed within the slot 130 of the angled insert 128, the lateral walls defining the slot 125 are positioned in planes parallel to those of the lateral walls defining the opening 130, with all of such planes forming oblique angles with the walls of the opening 108.

Similar to the insert 122, the system 100 may include one or more angled inserts 128 configured to accommodate different cutting instrumentation and having different sizing/dimensions/angulation. For example, a physician may desire to make an angled cut using an angled insert 128 implemented with the system 100 as shown in FIG. 15, and may select said angled insert 128 from a library of angled inserts 128 (with each angled insert 128 having an opening 130 forming a different oblique angle with the lateral walls defining the opening 108) provided with the system 100.

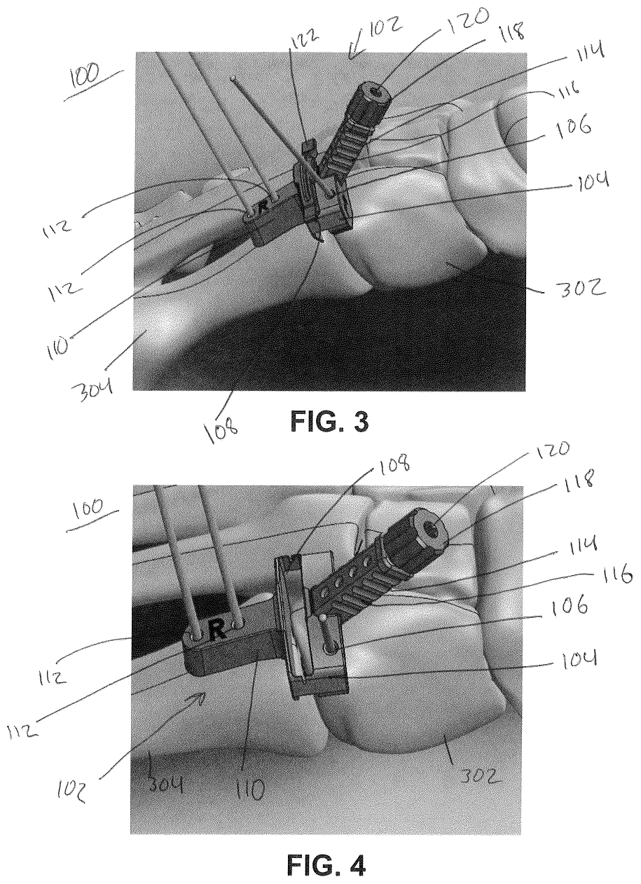

As shown in at least FIGS. 3 and 15, the system 100 may be implemented (with or without the angled insert 128) to provide guidance to a physician when making one or more cuts to the medial cuneiform 302 of a patient.

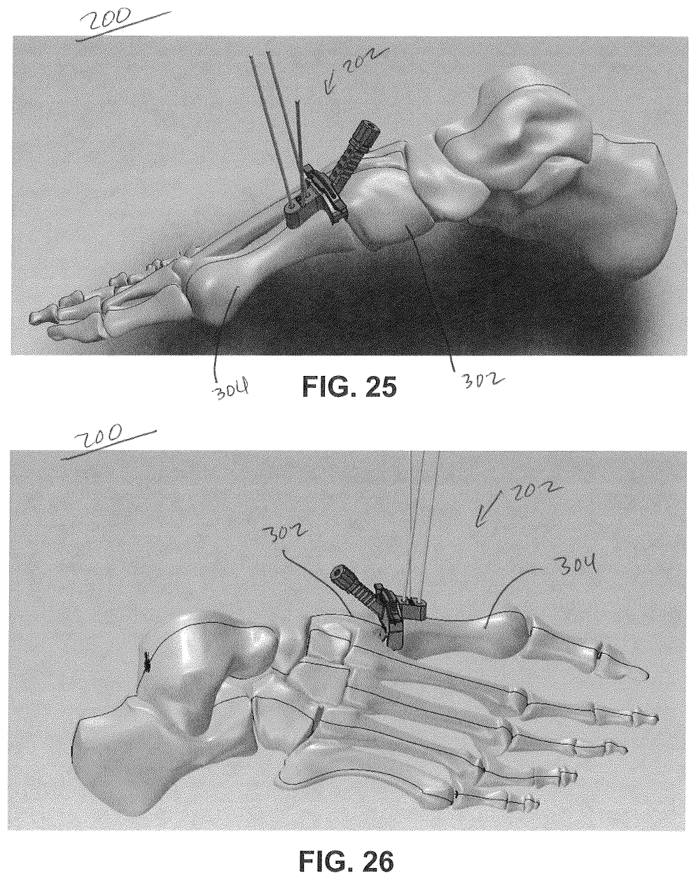

Referring now to FIGS. 25-38, the system 200 and components thereof are shown, according to an exemplary embodiment. The system 200 is shown to include a cutting guide 202 (referred to hereinafter as “guide 202”), which is shown to be coupled on distal side thereof with a first metatarsal 304 of a patient. The guide 202 may be releasably couplable with one or more bones of the anatomy, for example the medial cuneiform 302 and/or first metatarsal 304, by inserting one or more k-wires through openings in the guide 202 having a complimentary geometry to said k-wires, with the k-wires then advanced into the aforementioned bony anatomy.

The guide 202 is shown to include a base 204 which, as shown, includes a substantially rectangular footprint. In some aspects, the base 204 may include alternate geometries and, accordingly, alternate footprints (e.g., oval, pentagonal, hexagonal, etc.). In some aspects, the bottom surface of the base may include a curvature along at least a portion thereof configured to accommodate at least a portion of a contour of the first metatarsal 304 and/or the medial cuneiform 302. In some aspects, the base 204 may include one or more openings positioned thereon and extending therethrough the same as or similar to the opening 106 of the guide 102.

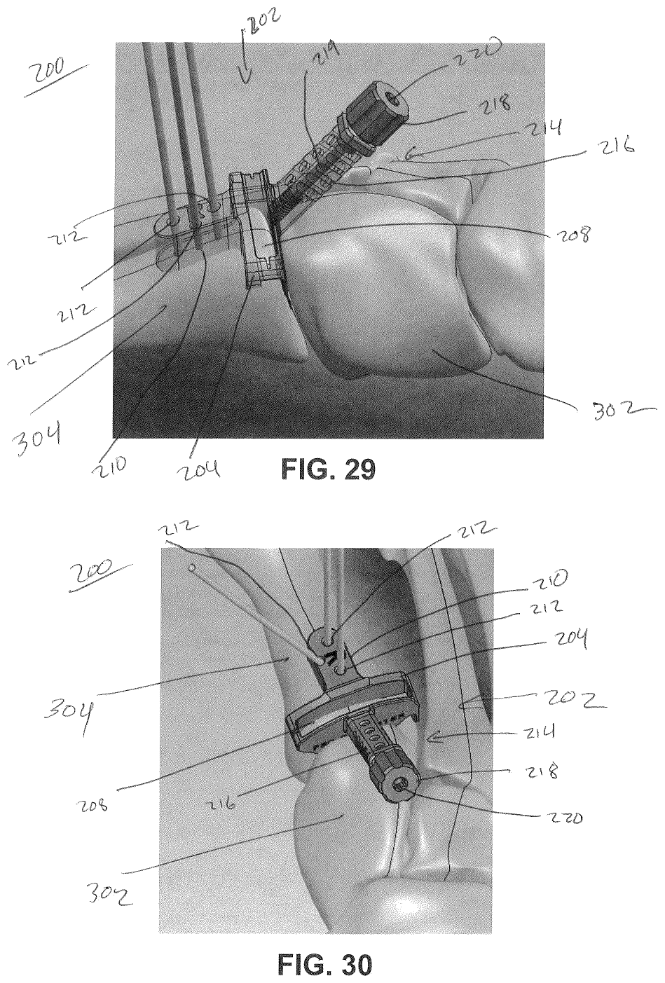

The base 204 is further shown to include an opening 208 positioned on a proximal portion thereof and extending from the top surface through to the bottom surface of the base 204 thus establishing fluid communication therebetween. In some aspects, the base 204 (and the opening 208 thereof) may be configured to receive one or more components therein (e.g., releasably couple with/retain said components). Further, in some aspects the opening 208 may be configured to receive at least a portion of a cutting instrument therein and therethrough, for example a saw (e.g., sagittal, reciprocating, etc.) or a burring instrument to guide cutting or otherwise modifying bony anatomy disposed beneath the opening 208. In some aspects, any such cutting or burring instruments may be manipulated through one or more slots or other openings disposed in the aforementioned inserts which may be releasably coupled with and/or retained within the opening 208. As shown, the opening 208 is positioned relative to the base 204 and the guide 202 such that, when the guide 202 is positioned as shown in at least FIG. 29, the opening 208 is positioned substantially above the first tarsometatarsal joint (and more specifically, a proximal portion of the first metatarsal 304) which is the joint at which the medial cuneiform 302 articulates at a distal portion thereof with a proximal portion of the first metatarsal 304. However, the opening 208 may also be positioned elsewhere about the base 204 and the guide 202 to facilitate positioning of the opening 208 relative to various bony anatomy or joints.

The base 204 includes a paddle 205 extending in a downward direction from the bottom surface of the base 204 just proximally relative to the opening 208. As shown, the paddle 205 is positioned at a proximal-most portion of the base 204 and may have a substantially rounded, triangular geometry (e.g., similar to that of a guitar pick) or similar. As shown in at least FIG. 29, the paddle 205 may be positioned within the first TMT joint (e.g., between the medial cuneiform 302 and the first metatarsal 304 when the guide 202 is engaged with said anatomy as shown.

The base 204 is further shown to include a projection 210 extending from the base 204 in the distal direction (e.g., just above the paddle 205). As shown, the projection 210 has a substantially elongated geometry with a rounded end, although the projection 210 may have alternate geometries in some embodiments. The projection 210 is shown to include at least one opening 212 (shown in FIGS. 25-38 as three openings 212) extending from a top surface of the projection 210 through to the bottom surface and establishing fluid communication therebetween. The at least one opening 212 may be sized to receive at least one k-wire therein and therethrough to facilitate engagement of the projection 210 with bony anatomy (e.g., the first metatarsal 304, as shown). As shown in FIGS. 25-38, the three openings 212 are positioned variously about the projection 210 approximately equidistant one another, but in some aspects the openings 212 may be positioned otherwise. Further, the projection 210 may include one, two, or various other numbers of openings 212 positioned variously about the projection 210.

The base 204 is further shown to include an extension 214, which is shown to extend from a proximal-most portion of the base 204 at an oblique angle relative to the top surface of the base 204. In some aspects, the extension 214 may extend at an approximately 45-degree angle from the base 204, or may extend from the base 204 at other, alternate angles. The extension 214 as shown includes a substantially rectangular body, which includes a texture 216 disposed on at least a portion of the outer surfaces thereof to facilitate gripping of the extension 214 by a physician. At least a portion of the extension 214 is shown to be substantially cannulated such that it may receive at least a portion of an actuator 218 (and a shaft 219 of the actuator 218) therein.

The actuator 218 includes a head portion 220, with the shaft 219 extending from the head portion 220. Both the head portion 220 and the shaft 219 may include a cannulation extending along at least a portion of a common longitudinal axis thereof. The shaft 219 includes a threading disposed on at least a portion of the outer surface thereof, shown in FIG. 29 to be positioned on a distal portion of the shaft 219. In some aspects, the extension 214 may include a threading complimentary to that of the shaft 219 within the cannulation and positioned at a distal end of the extension 214. Accordingly, the actuator 218 may be threadably coupled (and decoupled) with the extension 214 by twisting the head portion 220 so as to engage or disengage the threading of the shaft 219 with that of the extension 214. The head portion 220 of the actuator 218 may include one or more interfaces configured to facilitate engagement with one or more instruments, for example a hexalobe driver. The head portion 220 may include one or more geometric features configured to facilitate such engagement, and an instrument (such as the hexalobe driver) may be implemented by a physician in order to manipulate (e.g., loosen/tighten, advance/withdraw, etc.) the actuator 218 within the extension 214.

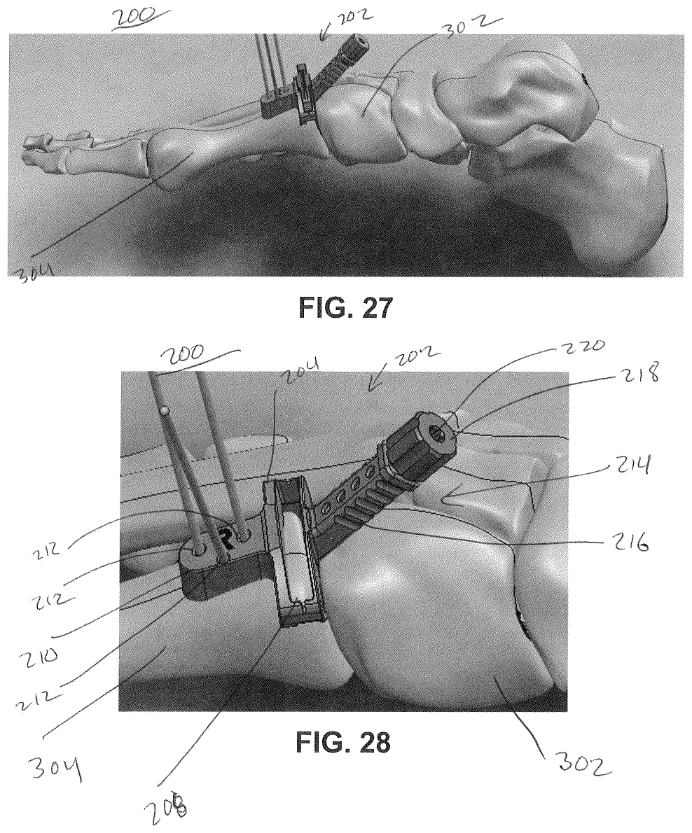

The system 200 is further shown to include an insert, as shown in FIG. 25, which may be the same as or similar to the insert 122. Similar to the engagement of the insert 122 with the guide 102, the insert 122 may be placed within the opening 208 of the guide 202 as shown in at least FIG. 27. When the insert 122 is positioned within the opening 208 (e.g., as shown in FIG. 27), the actuator 218 may be threadably coupled with the extension 214 and advanced via the threading of the shaft 219 along the extension 214 such that a distal-most portion of the shaft 219 contacts and is positioned within the depression 124. Accordingly, the insert 122 may be retained (e.g., in a coupled configuration) within the opening 208 by the actuator 218, as the distal portion of the shaft 219 prevents removal of the insert 122 from the opening 208 when the distal portion of the shaft 219 occupies at least a portion of the depression 124. The insert 122 may be decoupled from the guide 202 by manipulating the actuator 218 such that the distal portion of the shaft 219 vacates the depression 124, thus allowing for removal (e.g., decoupling) of the insert 122 from the opening 208.

Similar to the system 100, the system 200 may include one or more angled inserts 128 configured to accommodate different cutting instrumentation and having different sizing/dimensions/angulation. For example, a physician may desire to make an angled cut (to the first metatarsal 304) using an angled insert 128 implemented with the system 200 similar to that shown with respect to the system 100 in FIG. 15, and may select said angled insert 128 from a library of angled inserts 128 (with each angled insert 128 having an opening 130 forming a different oblique angle with the lateral walls defining the opening 208) provided with the system 200.

As shown in at least FIGS. 25-27, the system 200 may be implemented (with or without the angled insert 128) to provide guidance to a physician when making one or more cuts to the first metatarsal 304 of a patient.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An orthopedic instrument system, comprising:
a first cutting guide, comprising
a base portion comprising an opening;
an extension extending from the base portion comprising a cannulated opening in fluid communication with the opening of the base portion; and
an actuator configured to occupy at least a portion of the opening of the base when in an actuated position within the cannulated opening; and
a modular cutting insert configured to be received within the cannulated opening.

2. The orthopedic instrument system of claim 1, wherein the modular cutting insert comprises at least one depression.

3. The orthopedic instrument system of claim 2, wherein the at least one depression is disposed on a lateral surface of the modular cutting insert.

4. The orthopedic instrument system of claim 3, wherein the at least one depression comprises a volume which, when positioned within the opening, is common with at least a portion of the volume of the opening that is occupied by the actuator when the actuator is in the actuated position.

5. The orthopedic instrument system of claim 4, wherein the modular cutting insert comprises at least one slot positioned in a central portion of the modular cutting insert, with the at least one slot extending from a top surface through to a bottom surface of the modular cutting insert.

6. The orthopedic instrument system of claim 5, wherein the at least one slot is configured to receive at least a portion of an orthopedic instrument at least partially therein or therethrough.

7. The orthopedic instrument system of claim 6, wherein the modular cutting insert further comprises a first pair of protrusions extending from the top surface and each lateral surface of the modular cutting insert.

8. The orthopedic instrument system of claim 7, wherein the width between edges of the first pair of protrusions is greater than the width between lateral surfaces of the modular cutting insert.

9. The orthopedic instrument system of claim 7, wherein the modular cutting insert comprises a second pair of protrusions extending from the top surface and front and rear surfaces of the modular cutting insert.

10. The orthopedic instrument system of claim 9, wherein at least one of the first pair of protrusions and the second pair of protrusions are configured to contact an upper surface of the base portion adjacent the opening when the at least one slot is positioned within the opening.

11. The orthopedic instrument system of claim 10, further comprising a second modular cutting insert.

12. The orthopedic instrument system of claim 11, further comprising an angled insert.

13. The orthopedic instrument system of claim 12, wherein the angled insert comprises a length and width that is lesser than the length and width of the opening such that the angled insert is as least partially receivable within the opening.

14. The orthopedic instrument system of claim 13, wherein the angled insert comprises a central opening defined laterally by a first internal lateral surface and a second internal lateral surface, wherein at least one of the first and second internal lateral surfaces are positioned in planes that are oblique relative to a vertical axis.

15. An orthopedic instrument system, comprising:
a first cutting guide, comprising
a base portion comprising an opening;
an extension extending from the base portion comprising a cannulated opening in fluid communication with the opening of the base portion; and
an actuator configured to occupy at least a portion of the opening of the base when in an actuated position within the cannulated opening; and
a first modular cutting insert configured to be received within the cannulated opening.

16. The orthopedic instrument system of claim 15, wherein the first modular cutting insert comprises:
a body comprising a front surface, a rear surface, a top surface, a bottom surface, and a pair of opposing lateral surfaces;
a slot extending from the top surface through to the bottom surface;
a depression disposed on at least one of the lateral surfaces; and
a pair of protrusions extending from the top surface and at least one of the front and rear surfaces and the pair of lateral surfaces.

17. The orthopedic instrument system of claim 16, further comprising a second modular cutting insert.

18. The orthopedic instrument system of claim 17, wherein the second modular cutting insert comprises:
a body comprising a front surface, a rear surface, a top surface, a bottom surface, and a pair of opposing lateral surfaces;
a slot extending from the top surface through to the bottom surface;
a depression disposed on at least one of the lateral surfaces; and
a pair of protrusions extending from the top surface and at least one of the front and rear surfaces and the pair of lateral surfaces.

19. The orthopedic instrument system of claim 17, further comprising an angled insert.

20. An orthopedic instrument system, comprising:
a first cutting guide, comprising
a base portion comprising an opening;
an extension extending from the base portion comprising a cannulated opening in fluid communication with the opening of the base portion; and
an actuator configured to occupy at least a portion of the opening of the base when in an actuated position within the cannulated opening;
a first modular cutting insert configured to be received within the cannulated opening, the first modular cutting insert comprising:
a body comprising a front surface, a rear surface, a top surface, a bottom surface, and a pair of opposing lateral surfaces;
a slot extending from the top surface through to the bottom surface;
a depression disposed on at least one of the lateral surfaces; and
a pair of protrusions extending from the top surface and at least one of the front and rear surfaces and the pair of lateral surfaces; and
an angled insert.
